(19) United States
(12) Patent Application Publication
(10) Pub. No.: US 2013/0153474 A1
(43) Pub. Date: Jun. 20, 2013

(54) APPARATUS FOR EXTRACORPOREAL BLOOD TREATMENT, COMPRISING A MEASURING DEVICE FOR DETERMINING THE LUMINESCENCE OF THE SPENT DIALYSATE

(71) Applicants: Aleksandr Frorip, Elva (EE); Christoph Nacke, Tartu (EE); Stefan Moll, Hamburg (DE); Alex Castellarnau, Melsungen (DE); Joern Meibaum, Baunatal (DE)

(72) Inventors: Aleksandr Frorip, Elva (EE); Christoph Nacke, Tartu (EE); Stefan Moll, Hamburg (DE); Alex Castellarnau, Melsungen (DE); Joern Meibaum, Baunatal (DE)

(73) Assignee: B. Braun Avitum AG, Melsungen (DE)

(21) Appl. No.: 13/702,846

(22) PCT Filed: Jun. 10, 2011
(86) PCT No.: PCT/EP2011/059652

§ 371 Date: Jan. 29, 2013

(30) Foreign Application Priority Data
Jun. 11, 2010 (DE) 10 2010 023 486.9
Jun. 16, 2010 (EP) 10006210.8

(51) Int. Cl. A61M 1/16

(52) U.S. Cl. CPC A61M 1/1692
USPC 210/93; 210/97; 210/321.72

(57) ABSTRACT

The invention relates to an apparatus for extracorporeal blood treatment having a dialyzer which is divided into a first and a second chamber 29, 30 by means of a semipermeable membrane wherein the first chamber 29 is arranged in a dialysate path and the second chamber 30 can be connected to a patient's blood circulation by means of a blood supply line 32 and a blood discharge line 31, an inlet 20 for fresh dialysate, an outlet 36 for spent dialysate, a measuring device 37, wherein the measuring device 37 has at least one radiation source 1 for substantially monochromatic electromagnetic radiation. Thereby the measuring device 37 for determination of a luminescence of the spent dialysate flowing through the outlet 36 has at least one detector system (5) for detecting the intensity of the electromagnetic radiation generated by luminescence.

### CROSS REFERENCE TO RELATED APPLICATIONS

This application is the U.S. national phase application of PCT International Application No. PCT/EP2011/059652 filed Jun. 10, 2011, which claims priority to German Patent Application No. DE 10 2010 023 486.9 filed Jun. 11, 2010, and to European Patent Application No. EP 10006210.8 filed Jun. 16, 2010, the contents of each being incorporated by reference in their entirety.

### FIELD OF THE INVENTION

The invention relates to a method for determination of waste products during a dialysis treatment.

### BACKGROUND OF THE INVENTION

For patients with limited or lost renal function waste products of the natural metabolism including uraemic toxins are removed by dialysis methods, wherein the removal of the substances from the blood which is conducted extracorporeal occurs by contacting the blood with dialysate which carries various salts and thus causes diffusion and convective effects which are responsible for the transport of substances from blood into the dialysate via a membrane, wherein the purified blood is subsequently conveyed back to the patient.

The measure for the dialysis dose of a patient cannot only be made on the basis of the subjective evaluation of the health condition of the patient. It is necessary to quantify the success of dialysis in a way so that a sufficient effect of dialysis can be provided. Simultaneously a too high level of dialysis should also be avoided due to expenses. In order to render dialysis therapy more efficient it is necessary to control the dialysis efficiency real-time so that the therapy can be manually or automatically regulated by modification of the parameters of the dialysis machine.

In order to allow an adequate dialysis therapy a Kt/Vᵤᵣₑₐ model has been developed, wherein urea is one of waste products in the blood to be purified which is used for the determination of an adequate dialysis therapy and wherein K is the purification performance of the dialyzer for urea from the blood in ml/min, the treatment time in min and V the distribution volume of urea in ml in the human body which is directly correlated with the patient's weight. The nondimensional factor Kt/Vᵤᵣₑₐ defines the reduction of urea nitrogen in the blood upon treatment thrice a week.

The determination of the urea content or the content of toxic compounds in the dialysate outflow facilitates a comprehensive monitoring of the dialysis progress, however up to date thereto samples have to be taken manually from the system and transported to an appropriately equipped laboratory for chemical analysis.

Therefore the continuous monitoring of the haemodialysis was demanded (IEEE Engineering in Medicine & Biology Society 11^{th} international Conference) which is caused by the change in conductibility in the dialysate solution by hydrolysis of urea and/or other important molecules. Calibration of conductibility sensors which were specifically developed for this application however is very exhausting since influences on the conductibility can also arise from other sources. A measuring device which works according to this principle is the Biostat® Urea Monitor from Baxter.

Furthermore, the continuous monitoring of a haemodialysis can be put into practice by optical absorbance measurement which the transmission of the dialysate which is affected substantially by urea and other low molecular compounds. Such a measurement device is described by the UV-Monitoring System from Fridolin e.g. in EP1083948 B1.

Besides the reduction of urea however the removal behavior of other compounds like middle sized molecules in the range of 500 to ca. 50000 Da is important for evaluation of the dialysis efficiency since these are relevant for the pathology of uraemia. To these belong among others small proteins and peptides such as â₂-microglobin; cystatin C, retinol binding protein (RBP) or for example complement factor D (VFD). However, with those device and methods as described above and known in the state of the art it is not possible to continuously monitor the content of these compounds during a dialysis treatment in the purified blood or in the outflow of the dialysate. However a statement on middle sized molecules such as â₂-microglobin or other peptide or small proteins which are significantly responsible for the progress of uraemia is thereby not possible.

### SUMMARY OF THE INVENTION

Aspects of the present invention provide an apparatus which continuously allows a statement on the content or a change in the content of middle sized molecules in the range of 500 up to ca. 50000 Da in the outflow of the dialysate. Upon this a conclusion could be drawn continuously during treatment on the purification performance of the therapy in view of these molecules or the removal rate of these molecules from the blood to be purified, thus in real-time.

Absorbance and luminescence measurements are analytical methods to study molecules in the electromagnetic spectral range. Luminescence measurements are usually applied for detection of proteins which have structures capable for excitation such as fluorophors. Besides proteins also some peptides and other chemical substances exhibit such capability to emit electromagnetic radiation on the basis of luminescence effects. Such substances also enter during metabolism circulation of mammals their blood and have to be removed in order to maintain an operative metabolism on a continuing basis. In case the renal function of a mammal is affected these substances have to be removed from the blood of the mammal by means of a renal replacement therapy.

By the example of fluorescence, certain effects of luminescence are exemplary described. Within the principle of fluorescence a substance is irradiated with monochromatic, electromagnetic radiation of a certain wave length. In case the substance comprises fluorophors and the irradiated photons have the molecule specific energy for excitation of electrons, then the photons are absorbed in that the electrons interchange from the ground state into an energetically higher state. After duration of approx. 1 μs which is typical for fluorescence effects the excited electron leaves its energetically higher state and returns to the ground state. Upon this it irradiates the excess energy in the form of photons of a specific wave length. This wave length is generally 20-50 nm above the excitation wave length and arises from non-radiation effects within the energy bands of the molecule. This difference in wave length is generally known as the Stokes-Shift [“Topics in Fluorescence Spectroscopy”, Vol. 1-11, Joseph R. Lakowicz]. Simultaneously however there are also compounds which exhibit a far greater difference between excitation and emission wave length. One example is here p-cresol. The excitation wave length of this molecule is ca. 300 nm, while the emission wave length is ca. 600 nm. Excitation and emission wave length are characteristic properties for a molecule. Accordingly, conclusions can be drawn on the substances within a liquid by analysis of the excitation and emission spectra at luminescence effects such as fluorescence.

The invention connects the method of a luminescence measurement and/or a combination of luminescence and absorbance measurement in an apparatus for renal replacement therapy for the determination of the ingredients of waste product in the dialysate outflow, wherein the determination of the concentration of a specific compound or a combination of compounds within the dialysate wastes is directly dependent on the composition of the outflowing dialysate which flows out of the dialyzer in a dialysis treatment.

The purpose of the invention serves primarily an easy and reliable determination of dialysate ingredients in the dialysate outflow during a dialysis treatment in order to allow the patient an adequate success of treatment without stressing him/her to a too high level (“over dialyzing”). At the same time it is possible by analysis of the measurement results which contain either only the results of optical fluorescence measurements or the results of the combination of optical fluorescence and absorbance measurements to adapt the treatment of the patient to his/her specific, individual requirements in that dialysis specific parameters such as type of the dialyzer, type of therapy, level of the dialysate flow and so forth are adapted in terms of success of the patient.

Further, the invention is intended to the implementation of an apparatus for exact determination of waste products and their amounts in the dialysate outflow during a haemodialysis. This invention allows the combination of application of a known technique and the use of a reliable medical-technical apparatus for determination of proteins/peptides (as for example â₂-microglobin) but also other compounds which are contained in the dialysate after passing through the dialyzer so that influence on dialysis parameter like selection of dialyzer, blood flow, dialysate flow, type of therapy and many more is allowed by the measurement results.

During a treatment carried out with an apparatus of the present invention the determination of the content of dialysates in the outflow of the dialyzer is now possible, wherein the measurement of the ingredients and the concentration of the ingredients or a combination of several ingredients also in the middle molecular range in the dialysate flowing out of the dialyzer during a dialysis treatment by means of a spectral study by means of luminescence or fluorescence method or by means of a combination of absorbance and luminescence or fluorescence method of the dialysate, in order to determine dialysis specific parameters such as blood flow, dialysate flow, ultra filtration rate, type of therapy, duration of therapy.

During the therapy the determination of ingredients or the ingredients within the dialysate in the outflow and/or their concentration also in the middle molecular range is now possible in real-time.

Also it is made possible during therapy in real-time to determine relative changes of one ingredient or of a compound mixture in the middle molecular range e.g. by means of a Kt/V_{middle molecules}.

The results of the spectral studies can be shown together with the specific measurement parameters such as excitation wave length, emission wave length, and/or intensity profile of the emission wave length, in order to conclude on the content of a compound or a compound mixture in the dialysate flowing out of the dialyzer.

Also, the results of the spectral studies can be shown with dialysis parameters such as dialysate flow, blood flow, dialyzer etc. in order to conclude on the content of a compound or a compound mixture on the blood side of the dialyzer.

There upon the measurement of the dialysate can be carried out continuously, regularly or sporadic during a dialysis therapy.

Preferably the spectral photometric measurement is carried out by means of a UV-light emitting radiation source since the excitation energies of most of the substances to be determined are in the UV range.

Preferably, excitation wave lengths are used for the fluorescence measurement in the range of 1 to 750 nm.

Further, it was found to be advantageous that now an evaluation and a change or adaptation of the patient individual treatment parameters can be made on the basis of the analysis of the measurement results of fluorescence measurement or the combination of fluorescence and absorbance measurement of previous therapies of the same patient and/or of the same operating staff of the apparatus in real-time.

Preferably, therefore treatment parameters of the respective removal behavior (relative changes), the reduction rate and/or the dialysis dose Kt/V of one or several uraemic compounds or mixture of compounds are used.

The interesting uraemic compounds are thereby proteins and/or peptides in the middle molecular range of ˜500 up to ˜50000 Da such as â₂-microglobin or the like as well as small molecular substances with a molecular weight up to ˜500 Da such as uric acid, urea or the like.

In a preferred embodiment of the invention the measurement result or the measurement result in connection with afore-mentioned dialysis specific parameters is displayable on an output unit such as a screen and/or a printer.

According to a further idea of the invention one or several parameter(s) for the dialysis treatment depending on the measurement result or the measurement result in connection with other dialysis specific are modifiable, for example at reference data stored in a storage unit such as previous therapies of the same patient and/or of the same operating staff.

It was found explicitly advantageous, that the modification can be carried out automatically.

Alternatively, the modification can also be carried out manually.

It is particularly advantageous, if the parameter to be modified is the blood flow in the extracorporeal bloodstream, the ultra filtration rate, the dialysate flow, the type of therapy, the dialyzer and/or the duration of dialysis since these parameters have direct influence on therapy to be carried out.

In order to further automate a possible therapy, the inventive apparatus is designed for continuously carrying out the modification of one or several parameter(s) the dialysis therapy is carried out with depending on the measurement result.

### BRIEF DESCRIPTION OF THE DRAWINGS

The invention is best understood from the following detailed description when read in connection with the accompanying drawings. Included in the drawings are the following figures:

FIG. 1 is an inventive apparatus,

FIG. 2 is a progress of intensity over the wave length for the excitation and emission wave length of protein/peptide or other substances exemplary,

FIG. 3 is a preferred set up of a sensor for fluorescence measurement, and

FIG. 4 is a preferred set up of a combination of fluorescence and absorbance measurement.

### DETAILED DESCRIPTION OF THE PREFERRED EMBODIMENTS

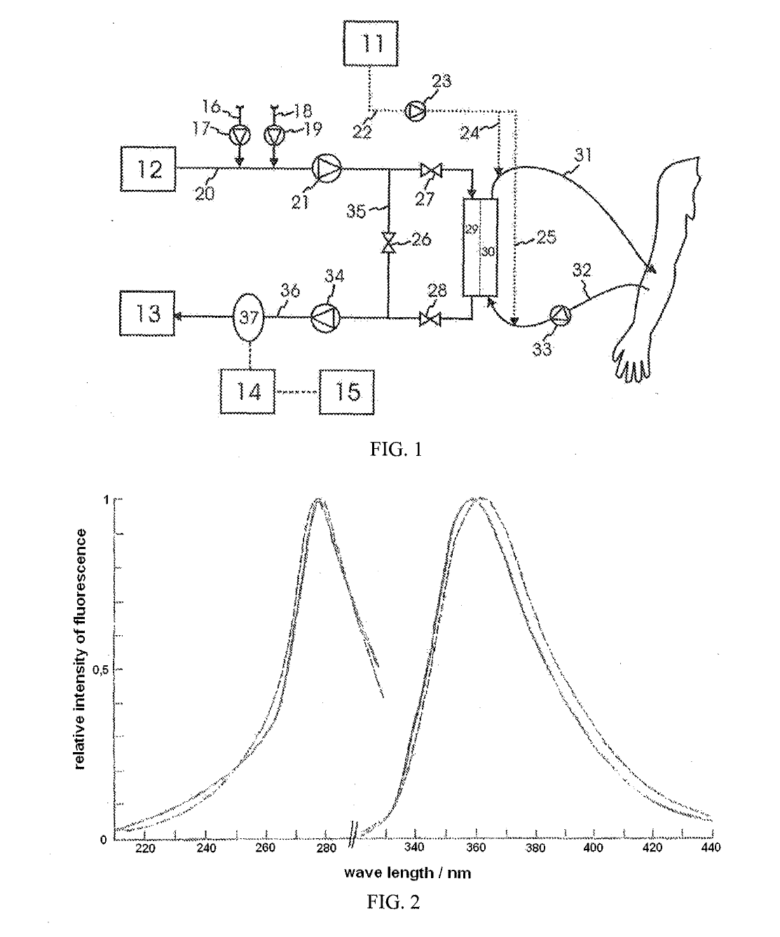

FIG. 1 shows the dialysate circuit of a conventional dialysis machine with an additional measuring device 37 which is arranged in the dialysate outlet 36. The blood of the patient is thereby led extracorporeal through a blood supply line 32 in the blood side chamber 30 of a dialyzer. After the blood has passed through the dialyzer it is led back to the patient via the blood discharge line 31. The blood flow is thereby controlled by blood pump 33. The dialysate consists of different ingredients which are dissolved in water. Thus the dialysis machine comprises a water inlet 12, two concentrate conduits 16, 18 and two concentrate pumps 17, 19. The water flow 20 determines together with the concentrate flow the composition of the dialysate. Via the water or the dialysate inlet 20 the dialysate is fed to the dialysate chamber 29 of the dialyzer which is divided from the blood chamber 30 by means of a semipermeable membrane. The dialysate is fed to the dialyzer by the dialysate pump 21 and is removed from the dialyzer via a further pump 34 together with the ultra filtrate. A by-pass connection is established between the pumps 21 and 34. Furthermore, valves 26, 27 and 28 are applied to control the dialysate circuit. Via hose segment 36 the dialysate is fed after passage through the dialyzer to the measuring device 37 which can carry out either a fluorescence measurement alone or a combination of fluorescence and absorbance measurement, where the optical measurements take place and sends the results subsequently via an interface to a corresponding analysis device 14. Subsequently the analyzed data is output via an output unit such as a screen and/or a printer. After the measurement the dialysate is fed to the outflow 13. The dashed lines 22, 24 and 25 represent the optional components of the system for carrying out a haemodial filtration. For that the substitution liquid is provided from the corresponding source 11 and is conveyed via the hose system 22 by means of a pump 23 in the blood circulation of the patient: In case of the option “post dilution” the substituate is fed to the blood circulation after passage through the dialyzer 24 and in case of the option “pre dilution” before the passage through the dialyzer 25. In case of the option “pre-post dilution” both ports 24 and 25 are used during feeding of the substituate. The computer 14 controls all elements which are shown in FIG. 1, and many more which have been left out for the sake of simplification. Furthermore the computer 14 derives information from the dialysis machine which can be mathematically connected with the results of the optical measurement device.

FIG. 2 shows a typical progress of intensity in dependence of excitation and emission wave length during a fluorescence measurement. A peak in intensity at low wave lengths corresponds thereby with intensity profile of excitation wave length while the increase of intensity at higher wave lengths shows the intensity profile of emission wave length. The shift of the maximum intensity of the excitation and emission wave length is called Stokes-Shift and relates back to non-radiating transitions within the molecule. The solid line describes the spectra for solid environmental conditions (pH, T, C etc.). Upon change of the environmental conditions (pH-value of the solved liquid, temperature, etc.) both spectra can change. That is indicated by the dashed line.

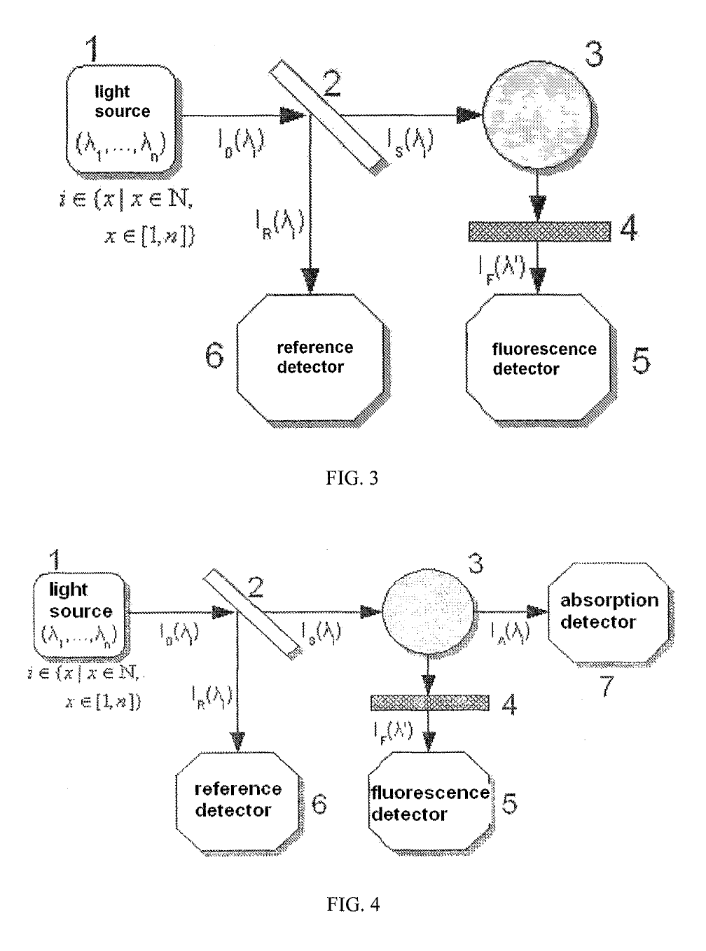

FIG. 3 shows the preferred set up of the measuring device 37 with fluorescence measurement. In principle every sensor unit for a luminescence measurement can be set up in the same way. Starting from a radiation source 1 electromagnetic radiation IO(λ) of a specific intensity and wave length profile, for example monochromatic light of the wave length λ=280 nm, is irradiated. This (light) is divided at an optical light beam divider 2. One part of the light is deflected to reference detector 6 and is there detected. The other part of the light which has passed unhindered the light beam divider 2 irradiates the sample to be studied in outlet 3 which shows the spent dialysate. Light emitted by fluorescence and luminescence effects leaves the one in outlet 3 and irradiates in all directions. A photo detector 5 which is arranged orthogonal to the original beam direction of the radiation source 1 detects the light being irradiated by the sample. For improved presentation of the emitted photons optical elements 4 such as optical grids or other filter units between outlet 3 and photodetector 5 are possible which in an ideal case are only permeable for the interesting wave length. By analysis of the wave length irradiated from the measurement sample conclusions can be drawn on specific ingredients such as β2-microglobin2-microglobin or other proteins/peptides and also on their concentration.

FIG. 4 shows the combination of luminescence or fluorescence and absorbance measurement for determination of dialysate ingredients in outlet 3 of the dialysate. The set up is similar to the one in FIG. 3, however this measurement unit is coupled with an additional absorbance detector 7 which in an ideal case lies in the optical axis of the radiation source 1. The combination of absorbance measurement upon simultaneous luminescence or fluorescence measurement enables the measurement of middle molecular compounds such as β2-microglobin2-microglobin upon simultaneous verification of the purification performance low molecular compounds by the absorbance measurement. Also here is the preferred wave length is λ=280 nm, wherein however it is variable in the range of 200 to ca. 400 nm. Otherwise the principle of measurement is strongly related to the principle of measurement in FIG. 3.

Besides the preferred embodiment of FIGS. 3 and 4 there are further embodiments possible which are not explicitly shown and which may also fall under the scope of the invention claimed. For example, the measuring device 37 can also be designed as a one beam photometer. For this, the elements 2 and 6 of FIGS. 3 and 4 are set aside. Also the radiation source 1 can be designed in various types. For example an embodiment as a monochromatic or also as a polychromatic radiation source is imaginable. Likewise the optical filter unit 4 can be designed as a bandpass with both a single pass range and also with multiple pass range. By variation of the radiation source 1 or the filter unit 4 molecules of different kind can be detected which enables an evaluation of the removal rate of toxic compounds from the blood.

In the described embodiment according to FIGS. 3 and 4 the measurement of the dialysate is carried out continuously over the treatment duration. However, it is also possible to carry out these measurements in defined time intervals. The operation of the measurements can be fully automated, but also a manual activation of the measurement is imaginable. The measurement process to be operated is either a fluorescence measurement alone like in FIG. 2 or a combination of fluorescence and absorbance measurement according to FIG. 3. The advantage of a combination resides in the fact to determine the reduction rate of low molecular compounds, which can be quiet well targeted with the absorbance measurement of EP 1 083 948 B1, parallel to the purification performance of middle molecular compounds, and to set in relation with the purification performance of middle molecular compounds which can be determined by the fluorescence measurement. From this combination conclusions cannot only be gathered on the current ingredients of the dialysate but also on the filter properties of the dialyzer, the purification progress of molecules of various molecular weights and toxicities and many more.

1-15. (canceled)

16. An apparatus for extracorporeal blood treatment comprising
a dialyzer which is divided into a first and a second chamber by means of a semipermeable membrane, wherein the first chamber is arranged in a dialysate path and second chamber is connected to a patient's blood circulation by means of a blood supply line and a blood discharge line,
an inlet for fresh dialysate,
an outlet for spent dialysate,
a measuring device that is arranged in the outlet, wherein the measuring device includes at least one radiation source for substantially monochromatic electromagnetic radiation, characterized in that
the measuring device for determination of a luminescence of the spent dialysate flowing through the outlet includes at least one detector system for detection of the intensity of the electromagnetic radiation generated by luminescence.

17. The apparatus according to claim 16, wherein that the radiation source for emission of electromagnetic radiation is designed in the range of 1 nm to 750 nm, particularly in the range of ultra-violet radiation of 1 nm to 380 nm.

18. The apparatus according to claim 16, wherein that the detector system for detection electromagnetic radiation is designed in the range of 1 nm to 750 nm.

19. The apparatus according to claim 16, wherein that the at least one radiation source for the substantially monochromatic electromagnetic radiation has a polychromatic radiation source and a monochromator, particularly an optical filter permeable for only a specific wave length or a bandpass filter with one or several pass ranges.

20. The apparatus according to claim 16, wherein that the at least one detector system for detection of the intensity of the electromagnetic radiation generated by luminescence has an optical filter permeable for only a specific wave length or a bandpass filter with one or several pass ranges.

21. The apparatus according to claim 16, wherein that the detector system is designed as a fluorescence detector.

22. The apparatus according to claim 16, wherein that the detector system with its optical axis is arranged substantially orthogonal to the optical axis of the radiation source.

23. The apparatus according to claim 16, wherein that the measuring device has a reference detector.

24. The apparatus according to claim 16, wherein that the measuring device has a light beam divider, which is arranged between radiation source and outlet.

25. The apparatus according to claim 16, wherein that the measuring device has an absorption detector which is preferably arranged in the optical axis of the radiation source, wherein the outlet is arranged between absorption detector and radiation source in their optical axis.

26. The apparatus according to claim 16, wherein that the radiation source is designed for pulsed or continuous emission of electromagnetic radiation.

27. The apparatus according to claim 16, wherein that it further has a microprocessor unit, a storage unit as well as an output unit.

28. The apparatus according to claim 27, wherein that the storage unit is designed for storage of reference data.

29. The apparatus according to claim 28, wherein that the microprocessor unit is designed for the modification of different apparatus parameters such as dialysate flow, blood flow, dialyzer or the like, by means of a comparison of the measured values with the reference data.

30. The apparatus according to one of claim 27, wherein that the measurement results provided by the detector system are displayable on the output unit, e.g. a printer or a monitor.

31. The apparatus according to one of claim 28, wherein that the measurement results provided by the detector system are displayable on the output unit, e.g. a printer or a monitor.

32. The apparatus according to one of claim 29, wherein that the measurement results provided by the detector system are displayable on the output unit, e.g. a printer or a monitor.
